(19) United States
(12) Patent Application Publication
(10) Pub. No.: US 2024/0066268 A1
(43) Pub. Date: Feb. 29, 2024

(54) MEDICAL DEVICES AND METHODS

(71) Applicant: George F. Wayne, Miami, FL (US)

(72) Inventor: George F. Wayne, Miami, FL (US)

(21) Appl. No.: 18/240,280

(22) Filed: Aug. 30, 2023

Related U.S. Application Data
(60) Provisional application No. 63/402,627, filed on Aug. 31, 2022.

(51) Int. Cl. A61M 25/02; A61F 5/453

(52) U.S. Cl. CPC A61M 25/02; A61F 5/453; A61M 2025/0206; A61M 2025/0213; A61M 2025/026; A61M 2210/1096

(57) ABSTRACT

In accordance with at least one aspect of this disclosure, a medical device can include a sheath having a proximal end and a distal end. The sheath can be configured to receive a penis through the proximal end, and can at least partially cover the penis. The sheath can include a distal opening in the distal end of the sheath. The sheath can include a flow structure comprising a proximal flow structure portion extending proximally relative to an interior surface of the distal end of the sheath and configured to be inserted into the urethra of the penis. The flow structure can be in fluid communication with or can pass through the distal opening in the distal end of the sheath to allow fluid to flow through the sheath to or from the urethra.

### CROSS-REFERENCE TO RELATED APPLICATIONS

This application claims priority to and the benefit of U.S. Provisional Application No. 63/402,627, filed Aug. 31, 2022, the entire contents of which are herein incorporated by reference in their entirety.

### FIELD

This disclosure relates to medical devices.

### BACKGROUND

Catheters are inserted into the penis of men for bladder drainage, urine collection, or certain medical procedures. Catheters may thus be inserted completely through the length of the urethra or only part-way for different purposes. In the past, male catheters were kept in place by taping the penis to the leg of the individual or by using accessory device to stabilize the penis while positioning the catheter as desired. Some of the most common problems with catheterization are from infection or contamination, or the catheter coming out.

Such conventional methods and systems have generally been considered satisfactory for their intended purpose. However, there is still a need in the art for improvements. The present disclosure provides a solution for this need.

### SUMMARY

In accordance with at least one aspect of this disclosure, a medical device can include a sheath having a proximal end and a distal end. The sheath can be configured to receive a penis through the proximal end, and can at least partially cover the penis. The sheath can include a distal opening in the distal end of the sheath. The sheath can include a flow structure comprising a proximal flow structure portion extending proximally relative to an interior surface of the distal end of the sheath and can be configured to be inserted into the urethra of the penis. The flow structure can be in fluid communication with or can pass through the distal opening in the distal end of the sheath to allow fluid to flow through the sheath to or from the urethra.

In certain embodiments, the flow structure can further include a distal flow structure portion that is in fluid communication with the proximal flow structure portion. The distal flow structure portion may be configured to extend distally from the distal end of the sheath. In certain embodiments the proximal portion and the distal flow structure portion can be a single piece or can be formed of a plurality of pieces. In certain embodiments, the distal flow structure portion of the flow structure can be configured to interface with a fluid connector or syringe.

In certain embodiments the distal flow structure portion can include an engagement mechanism to engage and/or lock to an external flow structure. In certain embodiments, the engagement mechanism can include a leuer-locking mechanism.

In certain embodiments, the distal flow structure portion can be configured to interface with a needle-less syringe. In certain embodiments, the proximal flow structure portion can include a catheter tip that can be configured to enter the urethral meatus of the penis. In certain embodiments, the proximal flow structure can be composed of a rigid or semi rigid material that can be configured to be inserted into the urethral meatus. In certain embodiments, the flow structure can be fused to the sheath.

In certain embodiments, the sheath can also comprise a circumferential retainer attached to the sheath on an outside of the sheath and configured to circumferentially fasten to maintain a position of the sheath axially on the penis and/or to provide traction to stretch the penis. In certain embodiments, the sheath can be a condom configured to at least partially cover the penis and to constrict around the penis. In certain embodiments, the sheath can be composed of an elastic material.

In certain embodiments, the circumferential retainer can be a strap including a hook and loop fastener that can be configured to allow selective tightening of the strap using the hook and loop fastener. In certain embodiments, the circumferential retainer can be positioned on the sheath in an axial location such that the circumferential retainer is configured to be tightened proximal to the glans of the penis and/or the corona glandis of the penis to create an axial stop.

In accordance with at least one aspect of this disclosure, a method can include inserting a flow structure that is attached to a sheath into a urethra of a penis through the urethral meatus of the penis. In certain embodiments, the method can include disposing the sheath over the penis. In certain embodiments, the method can include supplying fluid to the urethra through the flow structure.

In certain embodiments, the method can include connecting an external fluid supply to the flow structure to fluidly communicate the external fluid supply to the flow structure and the urethra. In certain embodiments, the method can include circumferentially tightening the sheath around the penis to axially fix the sheath to the penis. In certain embodiments, the method can include tightening the sheath by tightening a strap and fastening the strap. In certain embodiments, the method can include tightening the strap and fastening the strap proximal to the glans of the penis and/or the corona glandis of the penis to create an axial stop. In certain embodiments the method can include tightening an elastomeric penile clamp to maintain grip, stretch the penis, or create an axial stop.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain FIGURES, wherein:

FIG. 1 is a schematic elevation view of an embodiment of a medical device in accordance with this disclosure.

### DETAILED DESCRIPTION

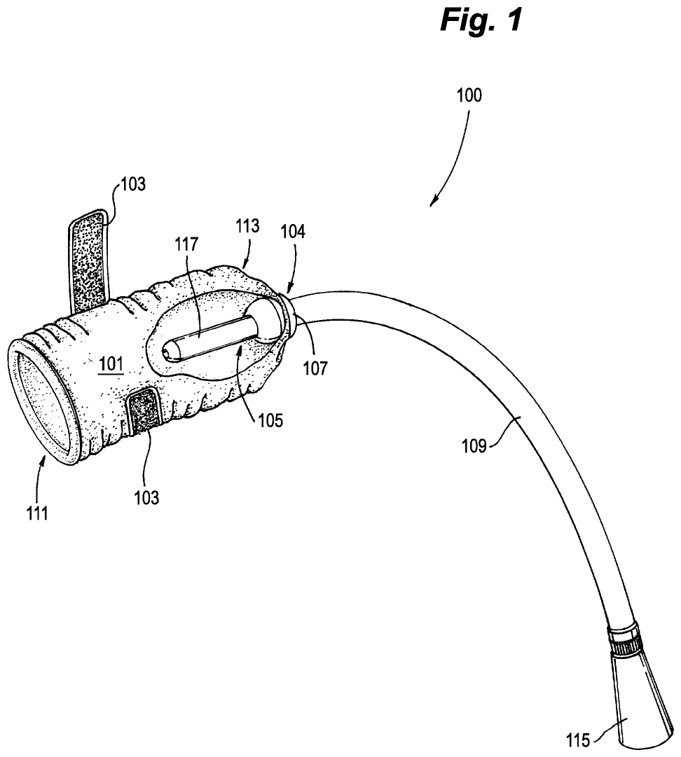

Reference will now be made to the drawings wherein like reference numerals identify similar structural features or aspects of the subject disclosure. For purposes of explanation and illustration, and not limitation, an illustrative view of an embodiment of a medical device in accordance with the disclosure is shown in FIG. 1 and is designated generally by reference character 100.

In accordance with at least one aspect of this disclosure, a medical device can include a sheath 101 having a proximal end 111 and a distal end 113. The sheath 101 can be configured to receive a penis through the proximal end 111, and can at least partially cover the penis for example. In certain embodiments the penis can enter the sheath 101 in the proximal end 111 and the sheath 101 can be pulled over the glans of the penis, or further down the shaft as deemed appropriate.

The sheath 101 can include a distal opening 107 in the distal end 113 of the sheath 101. The sheath 101 can include a flow structure 104 comprising a proximal flow structure portion 105 tending proximally relative to an interior surface of the distal end 113 of the sheath 101 and configured to be inserted into the urethra of the penis. For example, in certain embodiments, the proximal flow structure portion 105 can be a catheter 117 configured to enter the urethra of the penis.

The sheath 101 can function as a protective cover for the proximal flow structure portion 105, thereby reducing contamination and infection, and can also prevent movement of the proximal flow structure portion 105 once inserted into the penis. The flow structure 104 can be in fluid communication with or can pass through the distal opening 107 in the distal end 113 of the sheath 101 to allow fluid to flow through the sheath 101 to or from the urethra. For example, in certain embodiments, fluid can be inserted into the urethra of the penis through the proximal flow structure portion 105 (e.g., fluoroscopic fluids used to delineate the course of the urethra e.g., for a retrograde urethrogram). Any suitable fluids for insertion or removal are contemplated herein.

In certain embodiments, the flow structure can further include a distal flow structure portion 109 that is in fluid communication with the proximal flow structure portion 105. The distal flow structure portion 109 may be configured to extend distally from the distal end 107 of the sheath 101. For example, in certain embodiments the proximal flow structure portion 105 could be a catheter configured to enter the meatus of the penis, while the distal flow structure portion 109 extends distally for the release of urine from the penis, or can be used to insert material into the penis. In certain embodiments the proximal flow structure portion 105 and the distal flow structure portion 109 can be a single piece or can be formed of a plurality of pieces. For example, in certain embodiments the proximal portion 105 and/or the distal flow structure portion 109 could be made of multiple interchangeable pieces to be used for different procedures. In certain embodiments, the distal flow structure portion 109 of the flow structure can be configured to interface 115 with a fluid connector or syringe. For example, in certain embodiments of certain procedures, liquids could be inserted into the urethra from the syringe through the fluid connected.

In certain embodiments the distal flow structure portion 109 can include an engagement mechanism (not shown, e.g., a threaded end) to engage and/or lock to an external flow structure 115. In certain embodiments, the engagement mechanism (not shown) can include a leuer-locking mechanism. In certain embodiments, the distal flow structure portion 109 can be configured to interface with a needle-less syringe.

In certain embodiments, the proximal flow structure portion 105 can include a catheter tip 117 that can be configured to enter the urethral meatus of the penis. In certain embodiments, the proximal flow structure 105 can be composed of a rigid or semi rigid material that can be configured to be inserted into the urethral meatus. For example, in certain embodiments the proximal flow structure 105 could be made of a hard plastic or silicone. In certain embodiments, the flow structure 104 can be fused to the sheath 101. For example in certain embodiments fusing the flow structure 104 to the sheath could permit the sheath 101 to provide protection against contamination or infection, and the sheath 101 could also prevent movement of the flow structure 104 once inserted into the penis.

In certain embodiments, the sheath 101 can also comprise a circumferential retainer 103 attached to the sheath 101 on an outside of the sheath 101 and configured to circumferentially fasten to maintain a position of the sheath 101 axially on the penis and/or to provide traction to stretch the penis. In certain embodiments, the sheath 101 can be a condom configured to at least partially cover the penis and to constrict around the penis. For example, in certain embodiments the sheath 101 could be a condom that stretches over the penis, thereby providing flexibility in size and position, and also providing protection from contamination or infection. In certain embodiments, the sheath 101 can be composed of an elastic material. For example, in certain embodiments, the material could be made of a flexible watertight material such as rubber, plastic, or polyurethane. In certain embodiments, the circumferential retainer 103 can be a strap including a hook and loop fastener that can be configured to allow selective tightening of the strap using the hook and loop fastener. In certain embodiments, the circumferential retainer can be an elastomeric clamp (e.g., which can maintain grip, provide an axial stop, and place the penis on stretch, and which can be bottomed out to release its grip). In certain embodiments, the circumferential retainer 103 can be positioned on the sheath 101 in an axial location such that the circumferential retainer 103 is configured to be tightened proximal to the glans of the penis and/or the corona glandis of the penis to create an axial stop.

In accordance with at least on aspect of this disclosure, a method can include inserting a flow structure 105 that is attached to a sheath 101 into a urethra of a penis through the urethral meatus of the penis. In certain embodiments, the method can include disposing the sheath 101 over the penis. In certain embodiments, the method can include supplying fluid to the urethra through the flow structure 105.

In certain embodiments, the method can include connecting an external fluid supply to the flow structure 105 to fluidly communicate the external fluid supply to the flow structure and the urethra. In certain embodiments, the method can include circumferentially tightening the sheath 101 around the sheath 101 on the penis to axially fix the sheath 101 to the penis. In certain embodiments, the method can include tightening the sheath 101 by tightening a strap 103 and fastening the strap 103. In certain embodiments, the method can include tightening the strap 103 and fastening the strap 103 proximal to the glans of the penis and/or the corona glandis of the penis to create an axial stop.

Certain embodiments can include a device for assisting in the performance of a retrograde urethrogram. Certain embodiments can comprise a short condom, the tip of which is attached on the outside to rubber tubing and on the inside to a short (e.g., 16 F) catheter tip. In certain embodiments, the diameter can be reduced for narrower urethrae and/or can be manufactured in multiple urethral sizings for selection by the user based on penis and/or urethral size and length. The condom can also comprise a hook and loop fastener or an elastomeric penile clamp on the outside to be used as a fastener. Other suitable fastener types are contemplated herein. The condom can be placed on the patient's penis, with the catheter tip entering the meatus. The fastener can be tightened to maintain this positioning and to provide enough traction to stretch the penis. The rubber tubing emanating from the outside of the tip of the condom can interface bluntly with a needle-less syringe, which can be used to inject contrast (e.g., in a fluoroscopic setting this is used to delineate the course of the urethra and is referred to as a retrograde urethrogram).

The condom can be made from a flexible material such as elastic or rubber. The tubing can also be disconnected and either plugged-in or screwed into a leuer lock. The outer catheter can be detachable. The inner catheter can be fused to the condom, and can be guided into the tip of the urethra, then the condom portion can be unrolled, and the outside catheter can be attached. Certain embodiments can include a hook and loop fastener configured to tighten behind the glans or the ridge of the glans to create an axial stop.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A medical device comprising:
a sheath having a proximal end and distal end, wherein the sheath is configured to receive a penis through the proximal end to at least partially cover the penis, wherein the sheath includes a distal opening in the distal end;
a flow structure comprising a proximal flow structure portion extending proximally relative to an interior surface of the distal end of the sheath and configured to be inserted into the urethra of the penis, wherein the flow structure is in fluid communication with or passes through the distal opening in the distal end of the sheath to allow fluid to flow through the sheath to or from the urethra.

2. The medical device of claim 1, wherein the flow structure further comprises a distal flow structure portion in fluid communication with the proximal flow structure portion, wherein the distal flow structure portion is configured to extend distally from the distal end of the sheath.

3. The medical device of claim 2, wherein the proximal portion and the distal flow structure portion are a single piece or are formed of a plurality of pieces.

4. The medical device of claim 2, wherein the distal flow structure portion of the flow structure is configured to interface with a fluid connector or syringe.

5. The medical device of claim 2, wherein the distal flow structure portion includes an engagement mechanism to engage and/or lock to an external flow structure.

6. The medical device of claim 5, wherein the engagement mechanism includes a leuer-locking mechanism.

7. The medical device of claim 2, wherein the distal flow structure portion is configured to interface with a needle-less syringe.

8. The medical device of claim 1, wherein the proximal flow structure portion includes a catheter tip configured to enter the urethral meatus of the penis.

9. The medical device of claim 8, wherein the proximal flow structure portion is composed of a rigid or semi rigid material that is configured to be inserted into the urethral meatus.

10. The medical device of claim 1, wherein the flow structure is fused to the sheath.

11. The medical device of claim 1, further comprising a circumferential retainer attached to the sheath on an outside of the sheath and configured to circumferentially fasten to maintain a position of the sheath axially on the penis and/or to provide traction to stretch the penis.

12. The medical device of claim 1, wherein the sheath is a condom configured to at least partially cover the penis and to constrict around the penis.

13. The medical device of claim 1, wherein the sheath is composed of an elastic material.

14. The medical device of claim 1, wherein the circumferential retainer is a strap including a hook and loop fastener or elastomeric clamp configured to allow selective tightening of the strap using the fastener.

15. The medical device of claim 1, wherein the circumferential retainer is positioned on the sheath in an axial location such that the circumferential retainer is configured to be tightened proximal to the glans of the penis and/or the corona glandis of the penis to create an axial stop.

16. A method, comprising:
inserting a flow structure attached to a sheath into a urethra of a penis through the urethral meatus of the penis;
disposing the sheath over the penis; and
supplying fluid to the urethra through the flow structure.

17. The method of claim 16, further comprising connecting an external fluid supply to the flow structure to fluidly communicate the external fluid supply to the flow structure and the urethra.

18. The method of claim 16, further comprising circumferentially tightening the sheath around the penis to axially fix the sheath to the penis.

19. The method of claim 18, further comprising tightening the sheath by tightening a strap and fastening the strap.

20. The method of claim 19, further comprising tightening the strap and fastening the strap proximal to the glans of the penis and/or the corona glandis of the penis to create an axial stop.
